(19) United States
(12) Patent Application Publication
(10) Pub. No.: US 2007/0043423 A1
(43) Pub. Date: Feb. 22, 2007

(54) Intraluminal device with a hollow structure

(75) Inventor: David D. Grewe, West Lafayette, IN (US)

Correspondence Address: BRINKS HOFER GILSON & LIONE/CHICAGO/COOK, PO BOX 10395, CHICAGO, IL, 60610, US

(73) Assignee: MED Institute Inc., West Lafayette, IN (US)

(21) Appl. No.: 11/502,589

(22) Filed: Aug. 10, 2006

Related U.S. Application Data
(60) Provisional application No. 60/707,051, filed on Aug. 10, 2005.

(51) Int. Cl. A61F 2/06

(52) U.S. Cl. 623/1.11; 623/1.13; 623/1.42

(57) ABSTRACT

Intraluminal devices are provided with an inner cavity. The inner cavity may be loaded with a bioactive substance. Fenestrations extend between an outer surface and the inner cavity. Thus, the bioactive substance may be released from the intraluminal device through the fenestrations.

This application claims priority to U.S. Provisional Application No. 60/707,051, filed Aug. 10, 2005, which is hereby incorporated by reference herein.

### BACKGROUND

The present invention relates generally to medical devices and more particularly to intraluminal devices with a hollow structure.

A variety of intraluminal devices are known to those in the medical arts, including stents, stent-grafts, filters, occluders, artificial valves and other endoprosthetic devices. For example, stents have now become a relatively common device for treating a number of organs, such as the vascular system, colon, biliary tract, urinary tract, esophagus, trachea and the like. Stents are useful in a variety of medical procedures and are often used to treat blockages, occlusions, narrowing ailments and other related problems that restrict flow through a passageway. Stents are also useful in treating other ailments including various types of aneurysms.

Although stents and other medical devices are used in many different procedures, one common medical procedure in which stents are used involves implanting an endovascular stent into the vascular system. Stents have been shown to be useful in treating numerous vessels throughout the vascular system, including coronary arteries, peripheral arteries (e.g., carotid, brachial, renal, iliac and femoral), and other vessels. However, the use of stents in coronary arteries has drawn particular attention from the medical community because of the growing number of people suffering from heart problems associated with stenosis (i.e., a narrowing of an arterial lumen). This has lead to an increased demand for medical procedures to treat stenosis of the coronary arteries. In addition, the medical community has adapted many intravascular coronary procedures to other intraluminal disorders. The widespread frequency of heart problems may be due to a number of societal changes, including the tendency of people to exercise less while eating greater quantities of unhealthy foods, in conjunction with the fact that people generally now have longer life spans than previous generations. Stents have become a popular alternative for treating coronary stenosis because stenting procedures are considerably less invasive than other alternatives. Traditionally, stenosis of the coronary arteries has been treated with bypass surgery. In general, bypass surgery involves splitting the chest bone to open the chest cavity and grafting a replacement vessel onto the heart to bypass the blocked, or stenosed, artery. However, coronary bypass surgery is a very invasive procedure that is risky and requires a long recovery time for the patient.

Many different types of stents and stenting procedures are possible. In general, however, stents are typically designed as tubular support structures that may be inserted percutaneously and transluminally through a body passageway. Typically, stents are made from a metallic or other synthetic material with a series of radial openings extending through the support structure of the stent to facilitate compression and expansion of the stent. However, other types of stents are designed to have a fixed diameter and are not generally compressible. Although stents may be made from many types of materials, including non-metallic materials, common examples of metallic materials that may be used to make stents include stainless steel, nitinol, cobalt-chrome alloys, amorphous metals, tantalum, platinum, gold and titanium. Typically, stents are implanted within an artery or other passageway by positioning the stent within the lumen to be treated and then expanding the stent from a compressed diameter to an expanded diameter. The ability of the stent to expand from a compressed diameter makes it possible to thread the stent through narrow, tortuous passageways to the area to be treated while the stent is in a relatively small, compressed diameter. Once the stent has been positioned and expanded at the area to be treated, the tubular support structure of the stent contacts and radially supports the inner wall of the passageway. As a result, the implanted stent mechanically prevents the passageway from closing and keeps the passageway open to facilitate fluid flow through the passageway. However, this is only one example of how a stent may be used, and stents may be used for other purposes as well.

Particular stent designs and implantation procedures vary widely. For example, stents are often generally characterized as either balloon-expandable or self-expandable. However, the uses for balloon-expandable and self-expandable stents frequently overlap and procedures related to one type of stent are frequently adapted to other types of stents.

Balloon-expandable stents are frequently used to treat stenosis of the coronary arteries. Usually, balloon-expandable stents are made from ductile materials that plastically deform relatively easily. In the case of stents made from metal, 316L stainless steel which has been annealed is a common choice for this type of stent. One procedure for implanting balloon-expandable stents involves mounting the stent circumferentially on the balloon of a balloon-tipped catheter and threading the catheter through a vessel passageway to the area to be treated. Once the balloon is positioned at the narrowed portion of the vessel to be treated, the balloon is expanded by pumping saline through the catheter to the balloon. The balloon then simultaneously dilates the vessel and radially expands the stent within the dilated portion. The balloon is then deflated and the balloon-tipped catheter is retracted from the passageway. This leaves the expanded stent permanently implanted at the desired location. Ductile metal lends itself to this type of stent since the stent may be compressed by plastic deformation to a small diameter when mounted onto the balloon. When the balloon is later expanded in the vessel, the stent once again plastically deforms to a larger diameter to provide the desired radial support structure. Traditionally, balloon-expandable stents have been more commonly used in coronary vessels than in peripheral vessels because of the deformable nature of these stents. One reason for this is that peripheral vessels tend to experience frequent traumas from external sources (e.g., impacts to a person's arms, legs, etc.) which are transmitted through the body's tissues to the vessel. In the case of peripheral vessels, there is an increased risk that an external trauma could cause a balloon-expandable stent to once again plastically deform in unexpected ways with potentially severe and/or catastrophic results. In the case of coronary vessels, however, this risk is minimal since coronary vessels rarely experience traumas transmitted from external sources. In addition, one advantage of balloon-expandable stents is that the expanded diameter of the stent may be precisely controlled during implantation. This is possible because the pressure applied to the balloon may be controlled by the physician to produce a precise amount of radial expansion and plastic deformation of the stent.

Self-expandable stents are increasingly being used by physicians because of their adaptability to a variety of different conditions and procedures. Self-expandable stents are usually made of shape memory materials or other elastic materials that act like a spring. Typical metals used in this type of stent include nitinol and 304 stainless steel. However, other materials may also be used. A common procedure for implanting self-expandable stents involves a two-step process. First, the narrowed vessel portion to be treated may be dilated with an angioplasty balloon. Second, the stent is implanted into the portion of the vessel that has been dilated. Other variations are also possible, such as adding an additional dilation step after the stent has been implanted or implanting the stent without dilation. To facilitate stent implantation, the stent is normally installed on the end of a catheter in a low profile, compressed state. The stent is typically retained in the compressed state by inserting the stent into a sheath at the end of the catheter. The stent is then guided to the portion of the vessel to be treated. Once the catheter and stent are positioned adjacent the portion to be treated, the stent is released by pulling, or withdrawing, the sheath rearward. Normally, a step or other feature is provided on the catheter to prevent the stent from moving rearward with the sheath. After the stent is released from the retaining sheath, the stent radially springs outward to an expanded diameter until the stent contacts and presses against the vessel wall. Traditionally, self-expandable stents have been used in a number of peripheral arteries in the vascular system due to the shape memory characteristic of these stents. One advantage of self-expandable stents for peripheral arteries is that traumas from external sources do not permanently deform the stent. As a result, the stent may temporarily deform during unusually harsh traumas and spring back to its expanded state once the trauma is relieved. However, self-expandable stents may be used in many other applications as well.

The above-described examples are only some of the applications in which intraluminal devices are used by physicians. Many other applications for intraluminal devices are known and/or will be developed in the future. For example, similar procedures and treatments may also be applicable to vascular filters, occluders, artificial valves and other endoprosthetic devices.

The function of intraluminal devices may be enhanced in certain applications by adding a drug or other bioactive substance, which are referred to herein as medicants, to the intraluminal device. For example, in the case of stents, one problem that has been encountered with typical stenting procedures is restenosis (i.e., a re-narrowing of the vessel). Restenosis may occur for a variety of reasons, such as the vessel wall collapsing or the growth of new cellular tissue. For example, restenosis may occur as the result of damage caused to the vessel lining during balloon expansion and vessel dilation. This may cause the intima layers of the vessel to attempt to grow new intima tissue to repair the damage. The tendency of vessels to regrow new tissue may be referred to as neointimal hyperplasia. In addition, the synthetic materials that are usually used in stents may also contribute to neointimal hyperplasia. This is caused by the body's tendency to grow new living tissues around and over newly implanted foreign objects. The effect of these responses may result in a re-narrowing of the vessel. However, restenosis is not completely predictable and may occur either abruptly soon after the stenting procedure due to a collapse in the vessel or may occur slowly over a longer period of time for other reasons. In any event, restenosis may defeat the original purpose of the stenting procedure, which is generally to open a narrowed portion of a vessel and to maintain the patency of the vessel.

One approach that has been offered to address the problem of restenosis has been to coat stents with medicants that are designed to inhibit cellular growth. Although many such medicants are known, common examples of these types of medicants include Paclitaxel, Sirolimus and Everolimus. However, despite the benefits of these types of medicants, numerous problems still exist with the way that various medicants and other coatings are combined with stents and other intraluminal devices.

The simplest technique for combining beneficial medicants with an intraluminal device involves coating the medicant directly onto the outer surfaces of the device. Alternatively, various pits or reservoirs may be designed into the intraluminal device to receive the medicant. Common coating processes include dipping, spraying or painting the desired medicant onto the intraluminal device. However, current techniques for combining medicants with intraluminal devices suffer from numerous problems. For example, coatings that are applied to the surfaces of a device may be worn off before the device is implanted. As a result, only a portion of the medicant may remain on the device after implantation to serve the medicinal purpose. This may lead to an ineffective or non-uniform physiological response to the medicant that remains on the device. In addition, it may be desirable for the medicant to be released slowly to the surrounding tissues after implantation so that the effectiveness of the medicant may be maximized. However, it may be difficult to control the release of medicants applied to the outer surfaces of an intraluminal device since the coated surfaces of the device typically come into direct contact with the surrounding tissues or blood flow.

### SUMMARY

Intraluminal devices are described with a hollow structure. Fenestrations penetrate the wall of the hollow structure so that there is open communication between the outer surface of the structure and an inner cavity. A medicant may be loaded into the inner cavity and the fenestrations. As a result, once the intraluminal device is implanted, the medicant will be released to the surrounding tissues from the inner cavity through the fenestrations. Additional details and advantages are described below in the detailed description.

The invention may include any of the following aspects in various combinations and may also include any other aspect described below in the written description or in the attached drawings.

An intraluminal device is described that may include an implantable structure with at least a portion that is formed from a longitudinally extending hollow member which has an outer surface and an inner cavity extending longitudinally therethrough, where at least one fenestration extends through a wall of the hollow member between the inner cavity and the outer surface.

The intraluminal device may have opposing ends of the inner cavity that are closed. The hollow member of the intraluminal device may be a hollow tube. The intraluminal device may have a coating material adhered to the implantable structure, where the coating material covers the fenestration and thereby slows release of the medicant through the fenestration. The intraluminal device may include a rate controlling compound loaded into the inner cavity with the bioactive substance. The intraluminal device may include a rate controlling compound loaded into the fenestration and sealing the bioactive substance within the inner cavity, where the bioactive substance is diffusible through the rate controlling compound. The intraluminal device may include a medicant loaded into the inner cavity of the hollow member. The intraluminal device may be combined with a catheter which includes a distal end adapted to pass through a body cavity and a proximal end adapted to be manipulated in which the implantable structure is mounted on the distal end of the catheter and is deliverable through the body cavity. The implantable structure of the intraluminal device may be a stent structure that is formed from a series of structural members, where the hollow member includes at least one of the structural members, in which the stent structure is generally cylindrical with an inner diameter, an outer diameter, a proximal end, and a distal end, and a series of radial openings extend through the stent structure between the inner and outer diameters so that the stent structure expands from a compressed diameter to an expanded diameter. The stent structure of the intraluminal device may include a coil made from at least one of the hollow member, where the coil wraps around a circumference of the stent structure a multitude of times and extends along a length of the stent structure. The stent structure of the intraluminal device may include a mesh made from a plurality of the hollow members. The hollow members of the intraluminal device may be interleaved with each other. The hollow members of the intraluminal device may be physically adhered to each other at contact regions where the hollow members are disposed adjacent each other. The intraluminal device may include a stent structure that is self-expandable. The intraluminal device may include a stent structure that is balloon-expandable. The implantable structure may include an inner region directed toward an inner lumen and an outer region adapted to engage a vessel wall and the fenestration may open to one of the inner and outer regions and may be sized to release more of a bioactive substance to the one of the inner and outer regions than to the other of the inner and outer regions.

A method of treating an intravascular condition is described that may include accessing a vessel with an introduction catheter; passing a delivery catheter through the introduction catheter, the delivery catheter may include an intraluminal device mounted thereon, the intraluminal device may include a longitudinally extending hollow member having an outer surface and an inner cavity extending longitudinally therethrough, where at least one fenestration extends through a wall of the hollow member between the inner cavity and the outer surface, in which the inner cavity is loaded with a medicant; passing the delivery catheter through the vessel to a vessel portion to be treated; implanting the intraluminal device adjacent the vessel portion; and withdrawing the delivery catheter from the vessel and the introduction catheter.

The intraluminal device of the method may be a stent structure formed from a series of structural members, where the hollow member includes at least one of the structural members and the hollow member is a hollow tube, where opposing ends of the inner cavity are closed, and the stent structure is generally cylindrical with an inner diameter, an outer diameter, a proximal end, and a distal end, in which a series of radial openings extend through the stent structure between the inner and outer diameters to adapt the stent structure to expand from a compressed diameter to an expanded diameter. The medicant of the method may be an anti-restenosis medicant.

A method of manufacturing an intraluminal device is described that may include fabricating a structure from a hollow tube, where the hollow tube may include an outer surface and an inner cavity that extends longitudinally therethrough; penetrating a wall of the hollow tube to form a fenestration extending between the inner cavity and the outer surface; and loading a medicant into the inner cavity of the hollow tube.

The penetrating of the method may include using a laser to cut the fenestration through the wall of the hollow tube. The laser of the method may penetrate only one wall of the hollow tube without penetrating an opposing wall of the hollow tube. The laser of the method may penetrate both a first wall of the hollow tube and a second wall of the hollow tube opposing the first wall. The laser of the method may focus more energy on the first wall than on the second wall, where a first fenestration that extends through the first wall is formed larger than a second fenestration that extends through the second wall, such that a greater medicinal amount of the medicant elutes from the first fenestration than the second fenestration when the structure is implanted. The loading of the method may include dipping the structure in a fluid after the penetrating, where the fluid may include at least the medicant, and applying a vacuum to the fluid, such that the fluid passes through an open end of the inner cavity into the inner cavity. The structure of the method may be fully immersed in the fluid. The loading of the method may include dipping the structure in a fluid after the penetrating, where one end of the structure is immersed in the fluid and another end of the structure remains unimmersed, in which the fluid may include at least the medicant, and applying a vacuum to the fluid, such that the fluid passes between a first open end of the inner cavity immersed in the fluid and a second open end remaining unimmersed. The structure of the method may be a stent structure formed from a series of structural members, where the hollow tube may include at least one of the structural members, in which opposing ends of the inner cavity are closed, and the stent structure is generally cylindrical with an inner diameter, an outer diameter, a proximal end, and a distal end, where a series of radial openings extend through the stent structure between the inner and outer diameters to adapt the stent structure to expand from a compressed diameter to an expanded diameter. The loading of the method may include dipping the stent structure in a fluid after the penetrating, where the fluid may include at least the medicant, and applying a vacuum to the fluid, such that the fluid passes through an open end of the inner cavity into the inner cavity. The loading of the method may include mixing the bioactive substance with a solvent to raise a viscosity of the bioactive substance. The method may include loading a rate controlling compound into the inner cavity, where the inner cavity is loaded with both the bioactive substance and the rate controlling compound. The method may include loading the rate controlling compound into the inner cavity before loading the bioactive substance into the inner cavity. The loading of the bioactive substance in the method may include mixing the bioactive substance with a solvent to raise a viscosity of the bioactive substance, in which the bioactive substance has a higher affinity for the rate controlling compound than the solvent, the bioactive substance may be loaded into the inner cavity and the rate controlling compound at least in part by absorption. The method may include loading a rate controlling compound into the fenestration after the bioactive substance is loaded into the inner cavity, where the rate controlling compound may seal the bioactive substance within the inner cavity, in which the bioactive substance is diffusible through the rate controlling compound.

### BRIEF DESCRIPTION OF SEVERAL VIEWS OF THE DRAWINGS

The invention may be more fully understood by reading the following description in conjunction with the drawings, in which:

FIG. 1 is a perspective view of one embodiment of a stent;

FIG. 2 is a perspective view of another embodiment of a stent;

FIG. 3 is a perspective view of a stent-graft;

FIG. 4A is a cross sectional view of a hollow tube with fenestrations that penetrate through two walls of the tube;

FIG. 4B is a cross sectional view of a hollow tube with fenestrations that penetrate through only one wall of the tube;

FIG. 4C is a cross sectional view of a hollow tube with fenestrations that penetrate through two walls of the tube where the fenestrations are larger in one wall of the tube and smaller in the other wall of the tube;

FIG. 5 is an enlarged view of a mesh made from hollow tubes;

FIG. 6 is an enlarged view of two hollow tubes welded together where the hollow tubes contact each other; and

FIG. 7 is an illustration of a vacuum process for loading a fluid into the hollow tubes of a stent.

### DETAILED DESCRIPTION

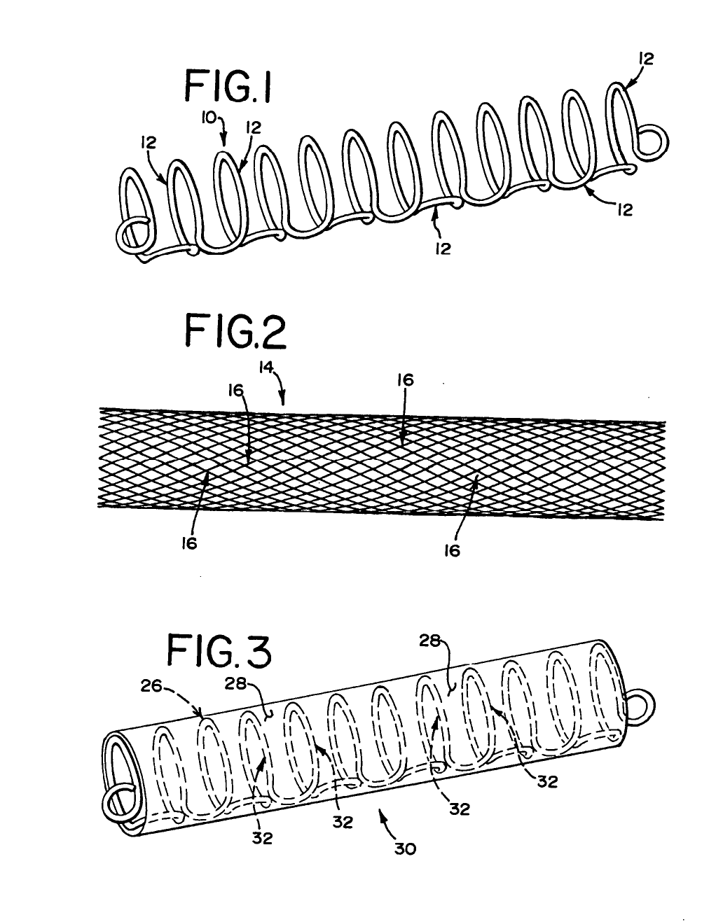

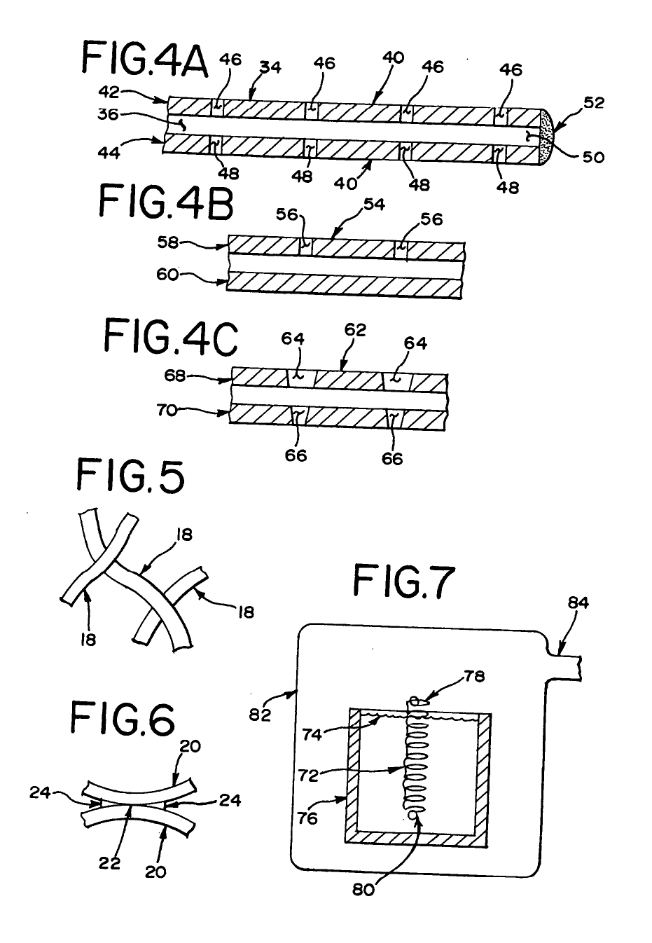

Referring now to the drawings, an endoluminal stent 10 is shown in FIG. 1. However, the invention may also be used with other intraluminal devices. As shown in detail in FIGS. 4A-4C and described further below, the stent 10 is made from a hollow wire 12 or tube. As shown in FIG. 1, the stent 10 is made from a single coiled wire 12 that is wrapped around the circumference of the stent structure multiple times along the length of the stent 10. Another type of stent structure is shown in FIG. 2. In FIG. 2, the stent 14 is made from a mesh of wires 16. As shown in FIGS. 5 and 6, the wires 18, 20 may interconnect with each other in a variety of ways. For example in FIG. 5, the wires 18 are interleaved with each other in an overlapping, braided manner. As shown in FIG. 6, the wires 20 may also be physically adhered to each other at contact regions 22 where portions of the wires 22 are physically adjacent each other. For example, as shown in FIG. 6, the wires 20 may be adhered to each other with a weld 24. However, the wires 20 may be adhered to each other in any manner that is known in the art including soldering, brazing, gluing or with other methods. Furthermore, the wires 18 shown in FIG. 5 may be physically adhered to each other in addition to being interleaved.

As shown in FIG. 3, a stent 26 may also be coated with a graft material 28 or other coating material. In the stent-graft 30 that is shown, the structural elements 32 of the stent 26 are encapsulated by the graft material 28. However, different arrangements are also possible. For example, the coating material may coat only a portion of the stent 26, such as the outer surfaces, or the coating material may coat only the structural elements 32 without bridging adjacent structural elements 32. Preferably, a soluble or permeable coating is used. For example, Thoralon or polyurethanes may be used. As described, further below, a coating that controls the release of a medicant is preferred.

Typically, stents are collapsible into a low profile configuration which is suitable for introducing the stent into a vessel of a patient and passing the stent through the vessel to a portion to be treated. This may be achieved using a variety of different procedures that may be adapted to particular intraluminal devices. For example, the stent may be mounted on the distal end of a delivery catheter. Where the stent is a balloon-expandable stent, the stent may be mounted on a balloon which contacts the inner surface of the stent. Where the stent is a self-expandable stent, the stent may be mounted within a retaining sheath which contacts the outer surface of the stent and retains the stent in the collapsed configuration. A patient's vessel may then be accessed using techniques that are well known to medical professionals. For example, a hollow needle may be used to penetrate the vessel, and a guide wire may be threaded through the needle into the vessel. The needle may then be removed and replaced with an introduction catheter. The introduction catheter generally serves the purpose of being a port which provides access to the vessel and through which various intraluminal tools and devices may be passed. The delivery catheter with the stent mounted thereon may then be passed through the introduction catheter and through the vessel to a vessel portion to be treated. Once the stent is positioned adjacent the vessel portion to be treated, the stent is implanted by either expanding the balloon or retracting the restraining sheath. This causes the stent to expand to its expanded configuration so that the outer surface of the stent contacts the vessel wall. The delivery catheter may than be withdrawn from the vessel and the introduction catheter. These techniques are not limited to stents, however, and may also be applicable to other intraluminal devices, such as vascular filters, occluders, artificial valves and other endoprosthetic devices.

In FIGS. 4A through 4C, hollow wires are shown that may be used to construct the stents shown in FIGS. 1 through 3. However, other hollow structures may also be possible. As shown in FIG. 4A, the hollow wire 34 has an inner cavity 36 that extends longitudinally along the length of the wire 34. Radially extending holes 46, 48, or fenestrations, extend from the outer surface 40 of the wire 34 to the inner cavity 36. Thus, there is open communication between the outer surface 40 of the wire 34 and the inner cavity 36. As shown in FIG. 4A, the holes 46, 48 may extend through both the top wall 42 and the bottom wall 44 of the wire 34, and the top holes 46 and the bottom holes 48 may be approximately equal in size. Although various structures and sizes are possible, a wire with a 0.005″ outer diameter and a wall thickness of 0.002″ may be used. Thus, the inner cavity of the hollow wire 34 may be as small as 0.001″. As described further below, it may be desirable to close the end 50 of the inner cavity 36. This may be accomplished with a plug 52 or by welding, soldering or brazing or may be accomplished in other ways. As shown in FIG. 4B, the holes 56 may penetrate only one of the walls 58 instead of both walls 58, 60 of the wire 54. For example, the holes 56 may penetrate only the top wall 58 but may not penetrate the bottom wall 60. In addition, as shown in FIG. 4C, the holes 64 penetrating one wall 68 may be different in size from holes 66 penetrating another wall 70.

The holes, or fenestrations, may be made in a number of ways. In addition, the fenestrations may have a variety of shapes and sizes. For example, the fenestrations may be holes as shown, but the fenestrations may also be slots or other shapes that penetrate from the outer surface of a hollow structure to an inner cavity. A preferred way to make the fenestrations is by using a laser. As shown in FIG. 4A, the laser may be used to penetrate all the way through the wire 34 to form holes 46, 48 of approximately the same size through both the top wall 42 and the bottom wall 44 of the wire 34. However, the type of laser, the energy intensity and/or the focal length may be adjusted so that the laser only penetrates the top wall 58 but not the bottom wall 60 as shown in FIG. 4B. Similarly, the laser may be adjusted so that it forms a larger hole 64 in the top wall 68 and a smaller hole 66 in the bottom wall 70 as shown in FIG. 4C. In addition to lasers, other methods may also be used to make the fenestrations, such as drilling holes with a mechanical drill. The fenestrations may also be made in the hollow structure before the intraluminal device is constructed or after the intraluminal device is constructed.

One benefit of the structures described above is that the hollow structures may be loaded with a medicant. As a result, the intraluminal device may release the medicant after the intraluminal device is implanted. This release may occur through the fenestrations from the inner cavity to the surrounding tissues or blood flow. For example, in the case of stents, anti-restenosis medicants like Paclitaxel, Sirolimus and Everolimus may have desirable physiological effects. Depending on the particular treatment, it may be desirable to load the inner cavity with other medicants or a combination of different medicants. For example, medicants that encourage specific tissue growth, such as VEGF growth factors, or which promote endothelium growth on the intraluminal device and on the damaged surrounding tissues may be desirable.

One advantage of loading medicants into the inner cavity of the hollow structures is that the inner cavity may retain these materials more securely and thereby release them more slowly over time. This may increase the length of time in which the medicant effectively treats the tissues. Furthermore, the medicant may be mixed with a diluent, such as dextran, in order to effectively slow release of the medicant from the intraluminal device. Moreover, the inner cavity and the fenestrations may have a larger capacity to store a greater quantity of a medicant compared with conventional medicant coatings. Moreover, the loaded medicants may also be less susceptible to being worn off the intraluminal device since the medicant is stored within the inner cavity and the fenestrations instead of directly on the outer surface of the device. This may result in a more reliable medicant treatment since the quantity of the medicant that is actually delivered to the tissues being treated may be more predictable. In addition, the hollow structures may be covered with a coating material that is soluble or permeable. This may aid in slowing the release of the medicant to provide a timed release. Moreover, depending on where the fenestrations are positioned, the medicant release may be directed toward specific tissues where the medicant is desired. For example, if it is desired to have the medicant released directly to a vessel wall but not to the inner lumen of the vessel, fenestrations on the outer surface of a stent but not the inner surface of the stent may be desirable. Such a structure may be constructed as shown in FIG. 4B. Alternatively, if more medicant is desired at the outer surface of a stent and a small amount of medicant is desired at the inner surface of the stent, a structure like that shown in FIG. 4C may be used. In general, it will be desirable to plug the open ends of the inner cavity as shown in FIGS. 4A-4B to slow the release of the medicant. In addition, the covered ends of the wires shown in FIGS. 4A-4B serve to provide a smooth end to prevent tissue damage which may occur from the blunt ends of a hollow wire.

The medicant may be loaded into the inner cavity and fenestrations in various ways. For example, the medicant may be pumped into the inner cavity with a pumping apparatus. However, a vacuum system is preferred. One vacuum system that may be used is shown in FIG. 7. As shown, a stent 72 may be immersed in a fluid 74 containing the medicant. The fluid 74 is held in a container 76. At this stage, the open ends of the inner cavities are preferably uncovered to facilitate fluid flow into the inner cavities through the ends. As shown in FIG. 7, one end 78 of the stent 72 may be positioned above the fluid 74 so that the top end 78 remains unimmersed. The bottom end 80 is immersed in the fluid 74. However, the entire stent 72 may also be immersed in the fluid. The fluid container 76 and the stent 72 may be placed in a vacuum vessel 82 to load the fluid 74 into the inner cavities and the fenestrations. Thus, as the vacuum source 84 is applied, the fluid 74 is drawn into the inner cavities through the open ends of the inner cavities and the fenestrations. After the inner cavities are filled with the medicant, the ends of the inner cavities are preferably plugged as described above. Depending on the desired use of the intraluminal device, the outer surfaces may or may not be covered by the medicant also. For example, in the vacuum process described above, the outer surfaces of the stent 72 will generally be coated by the medicant at the same time the inner cavities are loaded with the medicant. However, a masking agent may be used to cover the outer surfaces of the stent 72 to facilitate removal of the medicant from the outer surfaces if this is desired. Other techniques may also be used if it is desired to have the medicant only in the inner cavity or if other arrangements are desired. If another coating material is desired on the outer surface of the intraluminal device to slow the release of the medicant, this coating material may be applied by painting, dipping or spraying the outer surfaces of the device after the inner cavities have been loaded with the medicant.

It may also be desirable to mix the drug with a solvent or other compound to facilitate loading of the drug into the inner cavities. For example, various solvents that are well known may be used, such as dimethylacetamide (DMAC), tetrahydrofuran, alcohol, acetone or butylacetate. In general, a drug-solvent mixture may make it easier to load the drug into the inner cavities by raising the viscosity of the fluid mixture of the drug and the solvent. Such an approach may be desirable, for example, if the drug being used has a low viscosity at ambient temperatures and heating the drug in order to raise the viscosity is undesirable because of instability of the drug or other factors. If it is desirable to remove the solvent from the inner cavities after the drug has been loaded, the stent may be placed in a vacuum or heat oven.

In addition, the drug may also be combined with a rate controlling compound or polymer binder to control the release rate of the drug after the stent is implanted. Various compounds, which are known to those in the art, may be used to control the release of a drug, including polyurethane. The rate controlling compound may be directly mixed with the drug or drug-solvent mixture and loaded into the inner cavities as described above. Alternatively, the rate controlling compound may be loaded into the inner cavities or fenestrations before or after the drug is loaded into the inner cavities. For example, polyurethane may be loaded into the inner cavities first by melting the polyurethane or by any other conventional technique. The drug or drug-solvent mixture may then be loaded into the inner cavities and the polyurethane using a vacuum or heat oven. The drug may also be loaded into the polyurethane by absorption. For example, a polymer-drug-solvent combination could be used where the drug has a higher affinity for the polymer than the solvent. Thus, when the drug-solvent mixture is exposed to the polymer, the drug will absorb into the polymer. The rate controlling compound may also be loaded after the drug is loaded into the inner cavities to seal the fenestrations. As a result, the release of the drug may be slowed when the stent is implanted since the drug will be forced to diffuse through the rate controlling compound before contacting the surround tissues of the implantation site.

Desirably, an implantable medical device comprises a therapeutically effective amount of one or more therapeutic agents in pure form or in pharmaceutically acceptable salt, ester or prodrug form. Therapeutic agents that may be used in the present invention include, but are not limited to, pharmaceutically acceptable compositions containing any of the therapeutic agents or classes of therapeutic agents listed herein, as well as any salts and/or pharmaceutically acceptable formulations thereof. The implantable medical device can optionally comprise one or more therapeutic agents. Therapeutic agents for use in bio-compatible coatings include those known in the art. The bio-active agent of the present invention may include, for example, thrombo-resistant agents, antibiotic agents, anti-tumor agents, antiviral agents, anti-angiogenic agents, angiogenic agents, anti-mitotic agents, anti-inflammatory agents, angiostatin agents, endostatin agents, cell cycle regulating agents, genetic agents, including hormones such as estrogen, their homologs, derivatives, fragments, pharmaceutical salts and combinations thereof. Other useful bio-active agents include, for example, viral vectors and growth hormones such as Fibroblast Growth Factor and Transforming Growth Factor-β.

Medical devices comprising an antithrombogenic therapeutic agent are particularly preferred for implantation in areas of the body that contact blood. An antithrombogenic therapeutic agent is any therapeutic agent that inhibits or prevents thrombus formation within a body vessel. The medical device can comprise any suitable antithrombogenic therapeutic agent. Types of antithrombotic therapeutic agents include anticoagulants, antiplatelets, and fibrinolytics. Anticoagulants are therapeutic agents which act on any of the factors, cofactors, activated factors, or activated cofactors in the biochemical cascade and inhibit the synthesis of fibrin. Antiplatelet therapeutic agents inhibit the adhesion, activation, and aggregation of platelets, which are key components of thrombi and play an important role in thrombosis. Fibrinolytic therapeutic agents enhance the fibrinolytic cascade or otherwise aid is dissolution of a thrombus. Examples of antithrombotics include but are not limited to anticoagulants such as thrombin, Factor Xa, Factor VIIa and tissue factor inhibitors; antiplatelets such as glycoprotein IIb/IIIa, thromboxane A2, ADP-induced glycoprotein IIb/IIIa, and phosphodiesterase inhibitors; and fibrinolytics such as plasminogen activators, thrombin activatable fibrinolysis inhibitor (TAFI) inhibitors, and other enzymes which cleave fibrin.

Further examples of antithrombotic therapeutic agents include anticoagulants such as heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, CI-1083, JTV-803, razaxaban, BAY 59-7939, and LY-51,7717; antiplatelets such as eftibatide, tirofiban, orbofiban, lotrafiban, abciximab, aspirin, ticlopidine, clopidogrel, cilostazol, dipyradimole, nitric oxide sources such as sodium nitroprussiate, nitroglycerin, S-nitroso and N-nitroso compounds; fibrinolytics such as alfimeprase, alteplase, anistreplase, reteplase, lanoteplase, monteplase, tenecteplase, urokinase, streptokinase, or phospholipid encapsulated microbubbles; and other therapeutic agents such as endothelial progenitor cells or endothelial cells.

The therapeutic can also comprise one or more antibiotic agents. Antibiotic agents include penicillins, cephalosporins, vancomycins, aminoglycosides, quinolones, polymyxins, erythromycins, tetracyclines, chloramphenicols, clindamycins, lincomycins, sulfonamides their homologs, analogs, fragments, derivatives, pharmaceutical salts and mixtures thereof. Other therapeutic agents that can be utilized within the present invention include a wide variety of antibiotics, including antibacterial, antimicrobial, antiviral, antiprotozoal and antifungal agents. Representative examples of such agents include systemic antibiotics such as aminoglycosides (e.g. streptomycin, amikacin, gentamicin, netilmicin, tobramycin); 1st, 2nd, and 3rd generation cephalosporins (e.g., cephalothin, cefazolin, cephapirin, cephradine, cephalexin, cefadroxil, cefaclor, cefamandole, cefuroxime, cefuroxime axetil, cefonicid, ceforanide, cefoxitin, cefotaxime, cefotetan, ceftizoxime, cefoperazone, ceftazidime, ceftriaxone, moxalactam, other semisynthetic cephalosporins such as cefixime and cefpodoxime proxetil); penicillins (e.g., penicillin G (benzathine and procaine salts), clexacillin, dicloxacillin, methicillin, nafcillin, oxacillin, penicillin V, ampicillin, amoxicillin, bacampicillin, cyclacillin, carbenicillin, ticarcillin, meziocillin, piperacillin, azlocillin, amdinocillin, and penicillins combined with clavulanic acid); quinolones (e.g., cinoxacin, ciprofloxacin, nalidixic acid, norfloxacin, pipemidic acid, perloxacin, fleroxacin, enoxacin, ofloxacin, tosufloxacin, lomefloxacin, stereoisomers of the quinolones); sulfonamides (e.g., sulfacytine, sulfamethizole, sulfamethoxazole, sufisoxazole, sulfasalazine, and trimethoprim plus sulfamethoxazole combinations); tetracyclines (e.g., doxycycline, demeclocycline, methacycline, minocycline, oxytetracycline, tetracycline); macrolides (e.g., erythromycins, other semisythetic macrolides such as azithromycin and clarithromycin); monobactams (new synthetic class) (e.g., aztreonam, loracarbef); and miscellaneous agents such as actinomycin D, doxorubicin, mitomycin C, novobiocin, plicamycin, rifampin, bleomycin, chloramphenicol, clindamycin, oleandomycin, kanamycin, lincomycin, neomycin, paromomycin, spectinomycin, troleandomycin, amphotericin B, colistin, nystatin, polymyxin B, griseofulvin, aztreonam, cycloserine, clindamycin, colistimethate, imipenem-cilastatin, methenamine, metronidazole, nitrofurantoin, rifabutan, spectinomycin, trimethoprim, bacitracin, vancomycin, other β-lactam antibiotics.

Table 1 below provides a non-exclusive list of classes of various therapeutic agents and some corresponding exemplary active ingredients.

TABLE 1

| Therapeutic Agent | |
|---|---|
| Category | Exemplary Active Agents |
| Adrenergic agonist | Adrafinil; Isometheptene; Ephedrine |
| | (all forms) |
| Adrenergic antagonist | Monatepil maleate; Naftopidil; Carvedilol; |
| | Moxisylyte HCl |
| Adrenergic - | Oxymetazoline HCl; Norfenefrine HCl; |
| Vasoconstrictor/Nasal | Bretylium Tosylate |
| decongestant | |
| Adrenocorticotropic | Corticotropin |
| hormone | |
| Analgesic | Bezitramide |
| | Acetylsalicysalicylic acid |
| | Propanidid |
| | Lidocaine |
| | Pseudophedrine hydrochloride |
| | Acetominophen |
| | Chlorpheniramine Maleate |
| Anesthetics | Dyclonine HCl |
| | Hydroxydione Sodium |
| | Acetamidoeugenol |
| Anthelmintics | Niclosamide |
| | Thymyl N-Isoamylcarbamate |
| | Oxamniquine |
| | Nitroxynil N-ethylglucamine |
| | Anthiolimine |
| | 8-Hydroxyquinoline Sulfate |
| Anti-inflammatory | Bendazac |
| | Bufexamac |
| | Desoximetasone |
| | Amiprilose HCl |
| | Balsalazide Disodium Salt |
| | Benzydamine HCl |
| Antiallergic | Fluticasone propionate |
| | Pemirolast Postassium salt |
| | Cromolyn Disodium salt |
| | Nedocromil Disodium salt |
| Antiamebic | Cephaeline |
| | Phanquinone |
| | Thiocarbarsone |
| Antianemic | Folarin |
| | Calcium folinate |
| Antianginal | Verapamil |
| | Molsidomine |
| | Isosorbide Dinitrate |
| | Acebutolol HCl |
| | Bufetolol HCl |
| | Timolol Hydrogen maleate salt |
| Antiarryhythmics | Quinidine |
| | Lidocaine |
| | Capobenic Acid |
| | Encainide HCl |
| | Bretylium Tosylate |
| | Butobendine Dichloride |
| Antiarthritics | Azathioprine |
| | Calcium 3-aurothio-2-propanol-1-sulfate |
| | Glucosamine Beta Form |
| | Actarit |
| Antiasthmatics/Leukotriene | Cromalyn Disodium |
| antagonist | Halamid |
| | Montelukast Monosodium salt |
| Antibacterial | Cefoxitin Sodium salt |
| | Lincolcina |
| | Colisitin sulfate |
| Antibiotics | Gentamicin |
| | Erythromycin |
| | Azithromycin |
| Anticoagulants | Heprin sodum salt |
| | Heprinar |
| | Dextran Sulfate Sodium |
| Anticonvulsants | Paramethadione |
| | Phenobarbital sodium salt |
| | Levetiracetam |
| Antidepressants | Fluoxetine HCl |
| | Paroxetine |
| | Nortiptyline HCl |
| Antidiabetic | Acarbose |
| | Novorapid |
| | Diabex |
| Antiemetics | Chlorpromazine HCl |
| | Cyclizine HCl |
| | Dimenhydrinate |
| Antiglaucoma agents | Dorzolamide HCl |
| | Epinepherine (all forms) |
| | Dipivefrin HCl |
| Antihistamines | Histapyrrodine HCl |
| Antihyperlipoproteinemic | Lovastatin |
| | Pantethine |
| Antihypertensives | Atenolol |
| | Guanabenz Monoacetate |
| | Hydroflumethiazide |
| Antihyperthyroid | Propylthiouracil |
| | Iodine |
| Antihypotensive | Cortensor |
| | Pholedrine Sulfate |
| | Norepinephrine HCl |
| Antimalarials | Cinchonidine |
| | Cinchonine |
| | Pyrimethamine |
| | Amodiaquin Dihydrochloride dihydrate |
| | Bebeerine HCl |
| | Chloroquine Diphosphate |
| Antimigraine agents | Dihydroergotamine |
| | Ergotamine |
| | Eletriptan Hydrobromide |
| | Valproic Acid Sodium salt |
| | Dihydroergotamine mesylate |
| Antineoplastic | 9-Aminocamptothecin |
| | Carboquone |
| | Benzodepa |
| | Bleomycins |
| | Capecitabine |
| | Doxorubicin HCl |
| Antiparkinsons agents | Methixene |
| | Terguride |
| | Amantadine HCl |
| | Ethylbenzhydramine HCl |
| | Scopolamine N-Oxide Hydrobromide |
| Antiperistaltic; antidiarrheal | Bismuth Subcarbonate |
| | Bismuth Subsalicylate |
| | Mebiquine |
| | Diphenoxylate HCl |
| Antiprotozoal | Fumagillin |
| | Melarsoprol |
| | Nitazoxanide |
| | Aeropent |
| | Pentamideine Isethionate |
| | Oxophenarsine Hydrochloride |
| Antipsycotics | Chlorprothixene |
| | Cyamemazine |
| | Thioridazine |
| | Haloperidol HCl |
| | Triflupromazine HCl |
| | Trifluperidol HCl |
| Antipyretics | Dipyrocetyl |
| | Naproxen |
| | Tetrandrine |
| | Imidazole Salicylate |
| | Lysine Acetylsalicylate |
| | Magnesium Acetylsalicylate |
| Antirheumatic | Auranofin |
| | Azathioprine |
| | Myoral |
| | Penicillamine HCl |
| | Chloroquine Diphosphate |
| | Hydroxychloroquine Sulfate |
| Antispasmodic | Ethaverine |
| | Octaverine |
| | Rociverine |
| | Ethaverine HCl |
| | Fenpiverinium Bromide |
| | Leiopyrrole HCl |
| Antithrombotic | Plafibride |
| | Triflusal |
| | Sulfinpyrazone |
| | Ticlopidine HCl |
| Antitussives | Anethole |
| | Hydrocodone |
| | Oxeladin |
| | Amicibone HCl |
| | Butethamate Citrate |
| | Carbetapentane Citrate |
| Antiulcer agents | Polaprezinc |
| | Lafutidine |
| | Plaunotol |
| | Ranitidine HCl |
| | Pirenzepine 2HCl |
| | Misoprostol |
| Antiviral agents | Nelfinavir |
| | Atazanavir |
| | Amantadine |
| | Acyclovir |
| | Rimantadine HCl |
| | Epivar |
| | Crixivan |
| Anxiolytics | Alprazolam |
| | Cloxazolam |
| | Oxazolam |
| | Flesinoxan HCl |
| | Chlordiazepoxide HCl |
| | Clorazepic Acid Dipotassium salt |
| Broncodialtor | Epinephrine |
| | Theobromine |
| | Dypylline |
| | Eprozinol 2HCl |
| | Etafedrine |
| Cardiotonics | Cymarin |
| | Oleandrin |
| | Docarpamine |
| | Digitalin |
| | Dopamine HCl |
| | Heptaminol HCl |
| Cholinergic | Eseridine |
| | Physostigmine |
| | Methacholine Chloride |
| | Edrophonium chloride |
| | Juvastigmin |
| Cholinergic antagonist | Pehencarbamide HCl |
| | Glycopyrrolate |
| | Hyoscyamine Sulfate dihydrate |
| Congition | Idebenone |
| enhancers/Nootropic | Tacrine HCl |
| | Aceglutamide Aluminum Complex |
| | Acetylcarnitine L HCl |
| Degongestants | Propylhexedrine dl-Form |
| | Pseudoephedrine |
| | Tuaminoheptane |
| | Cyclopentamine HCL |
| | Fenoxazoline HCl |
| | Naphazoline HCl |
| Diagnostic aid | Disofenin |
| | Ethiodized Oil |
| | Fluorescein |
| | Diatrizoate sodium |
| | Meglumine Diatrizoate |
| Diuretics | Bendroflumethiazide |
| | Fenquizone |
| | Mercurous Chloride |
| | Amiloride HCl2H₂O |
| | Manicol |
| | Urea |
| Enzyme inhibitor | Gabexate Methanesulfonate |
| (proteinase) | |
| Fungicides | Candicidin |
| | Filipin |
| | Lucensomycin |
| | Amphotericin B |
| | Caspofungin Acetate |
| | Viridin |
| Gonad stimulating | Clomiphene Citrate |
| principle | Chorionic gonadotropin |
| | Humegon |
| | Luteinizing hormone (LH) |
| Hemorheologic agent | Poloxamer 331 |
| | Azupentat |
| Hemostatic | Hydrastine |
| | Alginic Acid |
| | Batroxobin |
| | 6-Aminohexanoic acid |
| | Factor IX |
| | Carbazochrome Salicylate |
| Hypolimpemic agents | Clofibric Acid Magnesium salt |
| | Dextran Sulfate Sodium |
| | Meglutol |
| Immunosuppresants | Azathioprine |
| | 6-Mercaptopurine |
| | Prograf |
| | Brequinar Sodium salt |
| | Gusperimus Trihydrochloride |
| | Mizoribine |
| Mydriatic; antispasmodic | Epinephrine |
| | Yohimbine |
| | Aminopentamide dl-Form |
| | Atropine Methylnitrate |
| | Atropine Sulfatemonohydrate |
| | Hydroxyamphetamine (I, HCl, HBr) |
| Neuromuscular blocking | Phenprobamate |
| agent/ | Chlorzoxazone |
| Muscle relaxants (skeletal) | Mephenoxalone |
| | Mioblock |
| | Doxacurium Chloride |
| | Pancuronium bromide |
| Oxotocic | Ergonovine Tartrate hydrate |
| | Methylergonovine |
| | Prostaglandin F_{2α} |
| | Intertocine-S |
| | Ergonovine Maleate |
| | Prostoglandin F_{2□} Tromethamine salt |
| Radioprotective agent | Amifostine 3H₂O |
| Sedative/Hypnotic | Haloxazolam |
| | Butalbital |
| | Butethal |
| | Pentaerythritol Chloral |
| | Diethylbromoacetamide |
| | Barbital Sodium salt |
| Serenic | Eltoprazine |
| Tocolytic agents | Albuterol Sulfate |
| | Terbutaline sulfate |
| Treatment of cystic fibrosis | Uridine 5′-Triphosphate Trisodium |
| | dihydrate salt |
| Vasoconstrictor | Nordefrin (−) Form |
| | Propylhexedrine dl-form |
| | Nordefrin HCl |
| Vasodilators | Nylidrin HCl |
| | Papaverine |
| | Erythrityl Tetranitrate |
| | Pentoxifylline |
| | Diazenium diolates |
| | Citicoline |
| | Hexestrol Bis(diethylaminoethyl ether) 2HCl |
| Vitamins | α-Carotene |
| | β-Carotene |
| | Vitamin D₃ |
| | Pantothenic Acid sodium salt |

Other desirable therapeutic agents include, but are not limited to, the following: (a) anti-inflammatory/immunomodulators such as dexamethasone, m-prednisolone, interferon g-1b, leflunomide, sirolimus, tacrolimus, everolimus, pimecrolimus, biolimus (such as Biolimus A7 or A9) mycophenolic acid, mizoribine, cyclosporine, tranilast, and viral proteins; (b) antiproliferatives such as paclitaxel or other taxane derivatives (such as QP-2), actinomycin, methothrexate, angiopeptin, vincristine, mitomycine, statins, C MYC antisense, ABT-578, RestenASE, Resten-NG, 2-chloro-deoxyadenosine, and PCNA ribozyme; (c) migration inhibitors/ECM-modulators such as batimastat, prolyl hydroxylase inhibitors, halofuginone, C proteinase inhibitors, and probucol; and (d) agents that promote healing and re-endotheliazation such as BCP671, VEGF, estradiols (such as 17-beta estradiol (estrogen)), NO donors, EPC antibodies, biorest, ECs, CD-34 antibodies, and advanced coatings.

Other suitable therapeutic agents include those described as bioactive agents in U.S. Patent Application Pub. No. 2004/0047909, which is incorporated herein by reference in its entirety.

A method of treating an intravascular condition is provided comprising: accessing a vessel with an introduction catheter; passing a delivery catheter through said introduction catheter, said delivery catheter comprising an intraluminal device mounted thereon, said intraluminal device comprising a longitudinally extending hollow member having an outer surface and an inner cavity extending longitudinally therethrough, at least one fenestration extending through a wall of said hollow member between said inner cavity and said outer surface, said inner cavity being loaded with a medicant; passing said delivery catheter through said vessel to a vessel portion to be treated; implanting said intraluminal device adjacent said vessel portion; and withdrawing said delivery catheter from said vessel and said introduction catheter.

Other aspects of the above-described method may include any combination of the following features. The method wherein said intraluminal device is a stent structure formed from a series of structural members, said hollow member comprising at least one of said structural members and said hollow member being a hollow tube, opposing ends of said inner cavity being closed, and said stent structure being generally cylindrical with an inner diameter, an outer diameter, a proximal end, and a distal end, a series of radial openings extending through said stent structure between said inner and outer diameters thereby adapting said stent structure to expand from a compressed diameter to an expanded diameter. The method wherein said medicant is an anti-restenosis medicant.

A method of manufacturing an intraluminal device is provided comprising: fabricating a structure from a hollow tube, said hollow tube comprising an outer surface and an inner cavity extending longitudinally therethrough; penetrating a wall of said hollow tube thereby forming a fenestration extending between said inner cavity and said outer surface; and loading a medicant into said inner cavity of said hollow tube.

Other aspects of the above-described method may include any combination of the following features. The method wherein said penetrating comprises using a laser to cut said fenestration through said wall of said hollow tube. The method wherein said laser penetrates only one wall of said hollow tube without penetrating an opposing wall of said hollow tube. The method wherein said laser penetrates both a first wall of said hollow tube and a second wall of said hollow tube opposing said first wall. The method wherein said laser focuses more energy on said first wall than on said second wall, a first fenestration extending through said first wall thereby being formed larger than a second fenestration extending through said second wall, whereby a greater medicinal amount of said medicant elutes from said first fenestration than said second fenestration when said structure is implanted. The method wherein said loading comprises dipping said structure in a fluid after said penetrating, said fluid comprising at least said medicant, and applying a vacuum to said fluid, whereby said fluid passes through an open end of said inner cavity into said inner cavity. The method wherein said structure is fully immersed in said fluid. The method wherein said loading comprises dipping said structure in a fluid after said penetrating, one end of said structure being immersed in said fluid and another end of said structure remaining unimmersed, said fluid comprising at least said medicant, and applying a vacuum to said fluid, whereby said fluid passes between a first open end of said inner cavity immersed in said fluid and a second open end remaining unimmersed. The method wherein said structure is a stent structure formed from a series of structural members, said hollow tube comprising at least one of said structural members, opposing ends of said inner cavity being closed, and said stent structure being generally cylindrical with an inner diameter, an outer diameter, a proximal end, and a distal end, a series of radial openings extending through said stent structure between said inner and outer diameters thereby adapting said stent structure to expand from a compressed diameter to an expanded diameter. The method wherein said loading comprises dipping said stent structure in a fluid after said penetrating, said fluid comprising at least said medicant, and applying a vacuum to said fluid, whereby said fluid passes through an open end of said inner cavity into said inner cavity. The method wherein said penetrating comprises using a laser to cut said fenestration through said wall of said hollow tube. The method wherein said loading comprises mixing said bioactive substance with a solvent, thereby raising a viscosity of said bioactive substance. The method further comprising loading a rate controlling compound into said inner cavity, said inner cavity thereby being loaded with both said bioactive substance and said rate controlling compound. The method further comprising loading said rate controlling compound into said inner cavity before loading said bioactive substance into said inner cavity. The method wherein said loading of said bioactive substance comprises mixing said bioactive substance with a solvent, thereby raising a viscosity of said bioactive substance, and wherein said bioactive substance has a higher affinity for said rate controlling compound than said solvent, said bioactive substance thereby being loaded into said inner cavity and said rate controlling compound at least in part by absorption. The method further comprising loading a rate controlling compound into said fenestration after said bioactive substance is loaded into said inner cavity, said rate controlling compound thereby sealing said bioactive substance within said inner cavity, said bioactive substance being diffusible through said rate controlling compound.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

I claim:

1. An intraluminal device, comprising:
an implantable structure comprising at least a portion formed from a longitudinally extending hollow member comprising a wall and an inner cavity extending longitudinally therethrough, at least one fenestration extending through said wall of said hollow member between said inner cavity and an exterior surface.

2. The intraluminal device according to claim 1, wherein opposing ends of said inner cavity are closed.

3. The intraluminal device according to claim 1, wherein said hollow member is a hollow tube.

4. The intraluminal device according to claim 1, further comprising a bioactive substance loaded into said inner cavity of said hollow member.

5. The intraluminal device according to claim 4, further comprising a coating material adhered to said implantable structure, said coating material covering said fenestration and thereby slowing release of said medicant through said fenestration.

6. The intraluminal device according to claim 4, further comprising a rate controlling compound loaded into said inner cavity with said bioactive substance.

7. The intraluminal device according to claim 4, further comprising a rate controlling compound loaded into said fenestration and sealing said bioactive substance within said inner cavity, said bioactive substance being diffusible through said rate controlling compound.

8. The intraluminal device according to claim 4, in combination with a catheter comprising a distal end adapted to pass through a body cavity and a proximal end adapted to be manipulated, wherein said implantable structure is mounted on said distal end of said catheter thereby being deliverable through said body cavity.

9. The intraluminal device according to claim 8, wherein said implantable structure is a stent structure formed from a series of structural members, said hollow member comprising at least one of said structural members, said stent structure being generally cylindrical with an inner diameter, an outer diameter, a proximal end, and a distal end, a series of radial openings extending through said stent structure between said inner and outer diameters thereby adapting said stent structure to expand from a compressed diameter to an expanded diameter.

10. The intraluminal device according to claim 9, wherein said stent structure comprises a coil made from at least one of said hollow member, said coil wrapping around a circumference of said stent structure a multitude of times and extending along a length of said stent structure.

11. The intraluminal device according to claim 9, wherein said stent structure comprises a mesh made from a plurality of said hollow members.

12. The intraluminal device according to claim 11, wherein said hollow members are interleaved with each other.

13. The intraluminal device according to claim 11, wherein said hollow members are physically adhered to each other at contact regions where said hollow members are disposed adjacent each other.

14. The intraluminal device according to claim 9, wherein said stent structure is self-expandable.

15. The intraluminal device according to claim 9, wherein said stent structure is balloon-expandable.

16. The intraluminal device according to claim 9, wherein said hollow member is a hollow tube and opposing ends of said inner cavity are closed.

17. The intraluminal device according to claim 16, further comprising a coating material adhered to said implantable structure, said coating material covering said fenestration and thereby slowing release of said bioactive substance through said fenestration.

18. The intraluminal device according to claim 1, wherein opposing ends of said inner cavity are closed, wherein said hollow member is a hollow tube, further comprising a coating material adhered to said implantable structure, said coating material covering said fenestration and thereby slowing release of said medicant through said fenestration, further comprising a rate controlling compound loaded into said inner cavity with said bioactive substance, further comprising a rate controlling compound loaded into said fenestration and sealing said bioactive substance within said inner cavity, said bioactive substance being diffusible through said rate controlling compound, further comprising a medicant loaded into said inner cavity of said hollow member, in combination with a catheter comprising a distal end adapted to pass through a body cavity and a proximal end adapted to be manipulated, wherein said implantable structure is mounted on said distal end of said catheter thereby being deliverable through said body cavity, wherein said implantable structure is a stent structure formed from a series of structural members, said hollow member comprising at least one of said structural members, said stent structure being generally cylindrical with an inner diameter, an outer diameter, a proximal end, and a distal end, a series of radial openings extending through said stent structure between said inner and outer diameters thereby adapting said stent structure to expand from a compressed diameter to an expanded diameter, wherein said stent structure comprises a coil made from at least one of said hollow member, said coil wrapping around a circumference of said stent structure a multitude of times and extending along a length of said stent structure, wherein said stent structure comprises a mesh made from a plurality of said hollow members, wherein said hollow members are interleaved with each other, and wherein said hollow members are physically adhered to each other at contact regions where said hollow members are disposed adjacent each other.

19. The intraluminal device according to claim 4, wherein said implantable structure comprises an inner region directed toward an inner lumen and an outer region adapted to engage a vessel wall, said fenestration opening to one of said inner and outer regions and being sized to release more of said bioactive substance to said one of said inner and outer regions than to the other of said inner and outer regions.

20. A method of manufacturing an intraluminal device, comprising:
fabricating a structure from a hollow tube, said hollow tube comprising an outer surface and an inner cavity extending longitudinally therethrough;
penetrating a wall of said hollow tube thereby forming a fenestration extending between said inner cavity and said outer surface; and
loading a medicant into said inner cavity of said hollow tube.
